(19) United States
(12) Patent Application Publication
(10) Pub. No.: US 2008/0269459 A1
(43) Pub. Date: Oct. 30, 2008

(54) Process for the Preparation of a Dicarboxylic Acid

(76) Inventors: Eit Drent, Amsterdam (NL); Rene Ernst, Amsterdam (NL); Willem Wabe Jager, Amsterdam (NL); Cornelia Alida Krom, Amsterdam (NL)

Correspondence Address: SHELL OIL COMPANY, P O BOX 2463, HOUSTON, TX, 772522463, US

(21) Appl. No.: 11/884,113

(22) PCT Filed: Feb. 10, 2006
(86) PCT No.: PCT/EP2006/050826

§ 371 Date: May 13, 2008

(30) Foreign Application Priority Data
Feb. 11, 2005 (EP) 05101023.9

(51) Int. Cl. C07C 57/13; C08G 73/00

(52) U.S. Cl. 528/363; 562/595

(57) ABSTRACT

A process for the preparation of a saturated dicarboxylic acid, comprising the steps of (a) contacting a conjugated diene with carbon monoxide and water to obtain a mixture containing an ethylenically unsaturated acid product and one or more reversible adducts of the conjugated diene and the ethylenically unsaturated acid; and (b) reacting the ethylenically unsaturated acid product further with carbon monoxide and water to obtain the dicarboxylic acid, wherein step (a) and (b) are performed in the presence of a catalyst system including a source of palladium, a source of an anion and a bidentate phosphine ligand, and wherein in step (a) the water concentration is maintained at a range of from 0.001 to less than 3% by weight of water, calculated on the overall weight of the liquid reaction medium, and wherein in step (b) the water concentration is maintained at a range of from 3% to 50% by weight of water, calculated on the overall weight of the liquid reaction medium.

### FIELD OF THE INVENTION

The present invention provides a process for the preparation of a dicarboxylic acid by carbonylation of a conjugated diene.

### BACKGROUND OF THE INVENTION

Carbonylation reactions of conjugated dienes are well known in the art. In this specification, the term carbonylation refers to a reaction of a conjugated diene under catalysis by a transition metal complex in the presence of carbon monoxide and water, as for instance described in WO 04/103948.

In WO 04/103948, a process is disclosed for the preparation of adipic acid from 1,3-butadiene or a mixture of 1,3-butadiene with olefinic products in a two-stage reaction. In the first stage of the disclosed process, 1,3-butadiene was reacted with carbon monoxide and water in the presence of a carbonylation catalyst comprising a palladium compound, a source of an anion and 1,2-bis(di-tert.-butyl-phosphino-methyl)benzene as bidentate diphosphine ligand for several hours until substantially all of the 1,3-butadiene was converted. To the obtained mixture comprising pentenoic acid product and the catalyst, in the second step additional water and carbon monoxide were added and the reaction was continued until at least part of the pentenoic acid product was converted to adipic acid. It was however found that both reaction steps were not very fast, which make the process less suitable for industrial applicability.

Accordingly, there remained the need to provide for a process for the preparation of saturated dicarboxylic acids with high turnover frequency in both carbonylation steps, thereby making the process suitable for industrial application.

It has now been found that the above identified process for the preparation of a saturated dicarboxylic acids product from a conjugated diene can be very effectively performed as set out below, which makes it particularly suited as a semi-continuous or continuous industrial scale process.

### SUMMARY OF THE INVENTION

Accordingly, the subject invention provides a process for the preparation of a dicarboxylic acid, comprising the steps of
(a) contacting a conjugated diene with carbon monoxide and water to obtain a mixture containing an ethylenically unsaturated acid product and reversible adducts formed by the conjugated diene with the ethylenically unsaturated acid; and
(b) reacting the ethylenically unsaturated acid with carbon monoxide and water to obtain the dicarboxylic acid;
   wherein steps (a) and (b) are performed in the presence of a catalyst system including a source of palladium, a source of an anion and a bidentate phosphine ligand, and wherein in step (a) the water concentration is maintained at a range of from 0.001 to less than 3% by weight of water, calculated on the overall weight of the liquid reaction medium, and wherein in step (b) the water concentration is maintained at a range of from 3% to 50% by weight of water, calculated on the overall weight of the liquid reaction medium.

### DETAILED DESCRIPTION OF THE INVENTION

Applicants found that the subject process permits to convert conjugated dienes into a dicarboxylic, preferably saturated dicarboxylic acid via an ethylenically unsaturated acid intermediate. Within the context of this specification, the terms “dicarboxylic acid” and “ethylenically unsaturated acid” may each describe a single compound or a mixture of isomers, depending on the structure of the conjugated diene employed. In the case of 1,3-butadiene as conjugated diene, the term ethylenically unsaturated acid describes 2-pentenoic acid, 3-pentenoic acid and 4-pentenoic acid and mixtures thereof, while the term “dicarboxylic acid” refers to adipic acid, and isomers of it such as 2-methyl-glutaric acid.

It was found that if the reaction steps (a) and (b) are performed in reaction medium of a very different polarity, in particular determined by the water concentration, each of these reaction steps is accelerated, increasing the overall reaction rate. It was in particular found that the overall reaction rate can be increased further if the concentration of the co-reactant water is chosen in such way, that in step (a) of the reaction only a small amount of water is constantly present, thereby creating an apolar reaction medium, whereas in step (b), a large amount of water is present, resulting in a more polar medium. The combination of these conditions resulted in a surprisingly high reaction speed in the respective carbonylation step. Accordingly, the subject reaction preferably makes use of the same catalyst system in both reaction steps. In step (a), the conjugated diene is contacted with carbon monoxide in the presence of 0.001 to less than 3% by weight of water, calculated on the overall weight of the liquid reaction medium, to obtain a mixture containing an ethylenically unsaturated acid product and one or more reversible adduct of the conjugated diene and the ethylenically unsaturated acid.

Subsequently, in step (b) the mixture obtained in step (a) is contacted with carbon monoxide in the presence of water in a range of from 3% to 50% by weight, calculated on the overall weight of the liquid reaction medium.

The catalyst may preferably be recycled from step (b) to step (a), with the proviso that surplus water is removed from the catalyst prior to recycling to step (a), or alternatively may be recycled from step (a) back to step (a), and from step (b) to step (b), thereby advantageously avoiding a water-removal step.

In step (a), the ratio (v/v) of conjugated diene and water in the feed can vary between wide limits and suitably lies in the range of 1:0.0001 to 1:500. However, it was found that the addition of water in step (a) to the reaction medium in order to provide a higher concentration of the reactant and hence an increased reaction rate had the opposite effect, i.e. an increase of the water concentration resulted in a strongly decreased reaction rate. Therefore, preferably, in step (a), less than 5% by weight of water is present in the reactor, yet more preferably, less than 3% by weight of water, yet more preferably, less than 1% by weight of water, again more preferably less than 0.15% by weight of water, and most preferably less than 0.01% by weight of water (w/w) is present in the reactor, calculated on the total weight of reactants. Again more preferably, these water concentrations are continuously present only, in particular if the reaction is performed as semi-batch or as continuous process. The water concentration may be determined by any suitable method, for instance by a Karl-Fischer-titration. It was equally found that the reaction speed of the reaction mixture may be influenced by other means, for instance by addition of an apolar solvent, e.g. toluene. At this point, part of the obtained mixture comprising the catalyst in admixture with the ethylenically unsaturated acid may be recycled to step (a), thus keeping a recycle with low water concentration.

In step (b), the mixture obtained in step (a) is pressurized again with carbon monoxide, and additional water is added as reactant for the carbonylation of the unsaturated acid product formed in step (a) is converted to a dicarboxylic acid under addition of carbon monoxide and water.

It was found that the reaction of the ethylenically unsaturated carboxylic acid to a dicarboxylic acid proceeds at an increased rate if the polarity of the medium is inverted to a highly polar medium, contrary to step (a). Therefore the water concentration throughout step (b) is higher than in step (a). Accordingly, the present invention relates to a process wherein in step (b) the water concentration in the reaction medium is maintained within the range of from to 3 to 50%, preferably from 4 to 30%, more preferably from 5 to 25%, and most preferably from 5 to 10% (w/w), based on the amount of the total liquid reaction medium.

Preferably, step (b) is performed as semi-batch or as continuous process, and more preferably, steps (a) and (b) are performed continuously.

In step (a) of the subject process, it was found that conjugated dienes have the tendency to reversibly form allylic alkenyl esters with any carboxylic acid present in the reaction mixture, in particular under catalysis by the carbonylation catalyst.

Depending on the reaction conditions, such alkenyl esters can be formed in substantial amounts.

Without wishing to be bound to any particular theory, it is believed that the formation of the esters from the conjugated diene and the ethylenically unsaturated acid is an equilibrium reaction catalyzed by the carbonylation catalyst, albeit at a comparatively slow rate. The presence of a high diene concentration, as well as an increasing amount of ethylenically unsaturated acid favours the formation of esters. In absence of catalyst, the equilibrium reaction becomes very slow, hence effectively freezing the equilibrium.

Since the alkenyl esters can be reverted into the conjugated diene and the ethylenically unsaturated acid, they are referred to as “reversible diene adducts” throughout the present specification. These “reversible diene adducts” were found to be remarkably stable in absence of the carbonylation catalyst. In the case of 1,3-butadiene as conjugated diene, the “reversible diene adducts” are butenyl esters with any suitable carboxylic acid present in the reaction mixture, thus mainly butenyl esters of 2-, 3- and 4-pentenoic acid, and mixtures thereof. Obviously, other acids present in the mixture may react with the conjugated diene, and thus may form reversible diene adducts as well.

In a preferred embodiment of the present process, step (a) of the present process is not allowed to proceed to full conversion of the conjugated diene and its reversible adducts, but is conducted only to a conversion of the conjugated diene of 99.95%, calculated on the amount of conjugated diene fed. Then conjugated diene and the reversible adducts are then preferably removed from the reaction mixture in an additional step (a1).

Without wishing to be bound to any particular theory, it is believed that this is due to the presence of unreacted diene, and reversible ester adducts formed at high diene concentrations, which only very slowly revert under catalysis by the palladium carbonylation catalyst to the conjugated diene and the acid to which they stand in equilibrium. Accordingly, the overall reaction rate becomes increasingly dependent on speed of the reversion of the reversible esters to conjugated diene. Only if substantially all conjugated diene has been converted, however, step (b) will achieve have a high initial carbonylation rate.

In the case of the carbonylation of 1,3-butadiene, step (a) is preferably allowed to proceed to 99% of conversion, based on moles of 1,3-butadiene converted versus moles of 1,3-butadiene fed. Yet more preferably, step (a) is allowed to proceed to 95% of conversion, again more preferably to 85% of conversion, again more preferably step to 65% of conversion, and yet more preferably step (a) is allowed to proceed to a range of from 30 to 60% of conversion. Then the conjugated diene and reversible diene adducts preferably are removed in process step (a1) from the reaction medium obtained in step (a).

In step (a1), carbon monoxide, conjugated diene, and the reversible ester products are removed from the reactor, while at least part of the ethylenically unsaturated acid product and the catalyst system remain in the reactor.

The removal of the reversible diene adducts in step (a1) may include the in-situ conversion of the remaining reversible diene adducts, and removal of the conjugated diene by stripping, or alternatively removal of the reversible diene adducts by distillate operation. The in-situ conversion is preferably done in the following manner: provided the conjugated diene is gaseous or has a low boiling point at ambient pressure, as for instance the case of 1,3-butadiene, the reaction mixture obtained in step (a) is brought near to atmospheric pressure, and then the conjugated diene is stripped from the reaction mixture under a gas flow, preferably comprising carbon monoxide to provide additional stability to the catalyst. In this way, the reversible diene adducts are forced to revert back into the conjugated diene and the ethylenically unsaturated acid, since constant removal of the conjugated diene with the gas stream will move the equilibrium towards reversion. The gaseous stream obtained in the stripping comprising carbon monoxide and conjugated diene may then advantageously be returned to step (a).

Alternatively, the reversible adducts are preferably removed from the reaction mixture in a distillative operation. The removed obtained ester mixture, usually also comprising some ethylenically unsaturated acid and by-products, is then either directly recycled to step (a), or converted in a separate conversion step in the presence of a suitable catalyst into conjugated diene and ethylenically unsaturated compound. At this point in the process, other undesired side-products can be preferably removed as well, such as vinyl cyclohexene in the case of 1,3-butadiene.

For a separate conversion step, the reversible diene adducts are contacted with a suitable catalyst before recycling the obtained conjugated diene and the unsaturated acid back to the process. Any catalyst suitable for the conversion may be applied, such as heterogeneous or homogeneous palladium catalysts. An example of a suitable palladium catalyst is the catalyst system as described for step (a) and (b). The reversible diene adducts usually have a boiling range below that of the unsaturated acid product.

The subject process permits to react conjugated dienes with carbon monoxide and a co-reactant. The conjugated diene reactant has at least 4 carbon atoms. Preferably the diene has from 4 to 20 and more preferably from 4 to 14 carbon atoms. However, in a different preferred embodiment, the process may also be applied to molecules that contain conjugated double bonds within their molecular structure, for instance within the chain of a polymer such as a synthetic rubber. The conjugated diene can be substituted or non-substituted. Preferably the conjugated diene is a non-substituted diene. Examples of useful conjugated dienes are 1,3-butadiene, 2-methyl-1,3-butadiene, conjugated pentadienes, conjugated hexadienes, cyclopentadiene and cyclohexadiene, all of which may be substituted. Of particular commercial interest are 1,3-butadiene and 2-methyl-1,3-butadiene (isoprene); most preferred being 1,3-butadiene in view of the commercial relevance of adipic acid.

In step (b), the mixture obtained in step (a), or optionally (a1) is pressurized again with carbon monoxide, and additional water is added as reactant for the carbonylation of the unsaturated acid product formed in step (a) to a dicarboxylic acid product.

In the case of the carbonylation of 1,3-butadiene, step (b) results in adipic acid product and in high purity. Adipic acid is a highly crystalline solid at ambient conditions. In the case that the process is conducted in pentenoic acid as solvent, adipic acid may begin to crystallize from the reaction mixture from a certain concentration and temperature onwards. If spontaneous crystallization in the reactor for step (b) is not desired, preferably, step (b) is only allowed to proceed until the liquid reaction medium comprises a saturated solution of adipic acid and/or any by-products at the reaction temperature in the liquid reaction medium.

Suitable sources of palladium for steps (a) and (b) include palladium metal and complexes and compounds thereof such as palladium salts; and palladium complexes, e.g. with carbon monoxide or acetyl acetonate, or palladium combined with a solid material such as an ion exchanger. Preferably, a salt of palladium and a carboxylic acid is used, suitably a carboxylic acid with up to 12 carbon atoms, such as salts of acetic acid, propionic acid and butanoic acid. A very suitable source is palladium (II) acetate.

Any bidentate diphosphine resulting in the formation of an active carbonylation catalyst with palladium may be used in the subject process. Preferably, a bidentate diphosphine ligand of formula R¹R²P—R—PR³R⁴ is employed, in which ligand R represents a divalent organic bridging group, and R¹, R², R³ and R⁴ each represent an organic group that is connected to the phosphorus atom through a tertiary carbon atom due to the higher activity found with such catalysts in both reaction steps. Yet more preferably, R represents an aromatic bidentate bridging group that is substituted by one or more alkylene groups, and wherein the phosphino groups R¹R²P— and —PR³R⁴ are bound to the aromatic group or to the alkylene group due to the observed high stability of these ligands. Most preferably R¹, R², R³ and R⁴ are chosen in such way, that the phosphino group PR¹R² differs from the phosphino group PR³R⁴. A very suitable ligand is 1,2-bis(di-tert.-butylphosphinomethyl)benzene. The ratio of moles of a bidentate diphosphine per mole atom of palladium preferably ranges from 0.5 to 50, more preferably from 0.8 to 10, yet more preferably from 0.9 to 5, yet more preferably in the range of 0.95 to 3, again more preferably in the range of 1 to 2, and yet most preferably it is stoichiometric. In the presence of oxygen, slightly higher than stoichiometric amounts of ligand to palladium are beneficial.

The source of anions preferably is an acid, more preferably a carboxylic acid, which preferably serves both as catalyst component as well as solvent for the reaction. Again more preferably, the source of anions is an acid having a pKa above 2.0 (measured in aqueous solution at 18° C.), and yet more preferably an acid having a pKa above 3.0, and yet more preferably a pKa of above 3.6. Examples of preferred acids include carboxylic acids, such as acetic acid, propionic acid, butyric acid, pentanoic acid, pentenoic acid and nonanoic acid, the latter three being highly preferred as their low polarity and high pKa was found to increase the reactivity of the catalyst system. 2-, 3- and/or 4-pentenoic acid are particularly preferred in case the conjugated diene is 1,3-butadiene, since this was found to not only form a highly active catalyst system, but also to be a good solvent for all reaction components.

The molar ratio of the source of anions, and palladium is not critical. However, it suitably is between 2:1 and 10⁹:1 and more preferably between 10⁷:1 and 10:1, yet more preferably between 10⁶:1 and 10²:1, and most preferably between 10⁵:1 and 10²:1 due to the enhanced activity of the catalyst system. Very conveniently the acid corresponding to the desired product of the reaction can be used as the source of anions in the catalyst. The process may optionally be carried out in the presence of an additional solvent, however preferably the intermediate acid product serves both as source of anions and as reaction solvent. Usually amounts in the range of 10⁻⁸ to 10⁻¹, preferably in the range of 10⁻⁷ to 10⁻² mole atom of palladium per mole of conjugated diene are used, preferably in the range of 10⁻⁵ to 10⁻² mole atom per mole of conjugated diene. In the case of 1,3-butadiene, it was found that if the amount of catalyst is chosen at a level below 20 ppm, calculated on the total amount of liquid reaction medium, side reactions, in particular Diels-Alder reactions of the conjugated diene, will become more prominent. In the case of 1,3-butadiene, side-products formed include 4-vinyl cyclohexene (further referred to as VCH, being the adduct of two 1,3-butadiene molecules), and most prominently, 2-ethyl cyclohexene carboxylic acid, further referred to as ECCA, which is the Diels-Alder adduct of 1,3-butadiene and 2-pentenoic acid. The formation of ECCA is favoured if 2-pentenoic acid also serves as a solvent. When 20 ppm of palladium catalyst were employed, ECCA was found to be formed in up to 3% by weight on total products. An increase of the catalyst concentration to 200 ppm is expected to result in a reduction of to 0.3% by weight of ECCA, and an increase of the catalyst concentration to 1000 ppm is expected to result in a reduction to 0.06% by weight of ECCA.

Accordingly, in steps (a) and (b), the carbonylation of 1,3-butadiene as conjugated diene is preferably performed in the presence of at least 20 ppm of catalyst, more preferably in the presence of 100 ppm of catalyst, and most preferably in the presence of at least 500 ppm. Although this requires a larger amount of palladium to be employed, the catalyst may advantageously be recycled from step (a) or (b) of the reaction of either step (a) or (b).

Examples of suitable catalyst systems as described above are those disclosed in EP-A-1282629, EP-A-1163202, WO2004/103948 and/or WO2004/103942. Most preferably, though, the reaction is performed in the ethylenically unsaturated acid products and/or the saturated dicarboxylic acids product, provided the mixture remains liquid at reaction conditions.

The carbonylation reaction according to the present invention in steps (a) and (b) is carried out at moderate temperatures and pressures. Suitable reaction temperatures are in the range of 0-250° C., more preferably in the range of 50-200° C., yet more preferably in the range of from 80-150° C.

The reaction pressure is usually at least atmospheric pressure. Suitable pressures are in the range of 0.1 to 25 MPa (1 to 250 bar), preferably in the range of 0.5 to 15 MPa (5 to 150 bar), again more preferably in the range of 0.5 to 9.5 MPa (5 to 95 bar) since this allows use of standard equipment. Carbon monoxide partial pressures in the range of 1 to 9 MPa (10 to 90 bar) are preferred, the upper range of 5 to 9 MPa being more preferred. Again higher pressures require special equipment provisions, although the reaction would be faster since it was found to be first order with carbon monoxide pressure.

In the process according to the present invention, the carbon monoxide can be used in its pure form or diluted with an inert gas such as nitrogen, carbon dioxide or noble gases such as argon, or co-reactant gases such as ammonia.

Process steps (a) to (b) are preferably performed in a continuous operation. Steps (a) and (b) of the subject process are suitably performed in a single reactor suitable for gas-liquid reactions, or a cascade thereof, such as constant flow stirred tank reactor, or a bubble column type reactor, as for instance described in “Bubble Column Reactors” by Wolf-Dieter Deckwer, Wiley, 1992. A bubble column reactor is a mass transfer and reaction device in which in one or more gases are brought into contact and react with the liquid phase itself or with a components dissolved or suspended therein. Preferably, a reactor with forced circulation is employed, which are generally termed “ejector reactors”, or if the reaction medium is recycled to the reactor, “ejector loop reactors”. Such ejector reactors are for instance described in U.S. Pat. No. 5,159,092 and JP-A-11269110, which employ a liquid jet of the liquid reaction medium as a means of gas distribution and circulation.

The dicarboxylic acid may be isolated from the reaction mixture by various measures. Preferably, the dicarboxylic acid is isolated from the reaction mixture by crystallization of the dicarboxylic acid in the reaction mixture and separation of the dicarboxylic acid crystals from the remaining reaction mixture containing the catalyst. It has been found that the dicarboxylic acid crystals can be obtained in a high purity in only a few crystallization steps, making it an efficient method for the separation of the product from the catalyst and unreacted ethylenically unsaturated acid intermediate. Accordingly, the subject process further preferably comprises a further process step of purifying the dicarboxylic acid. The process also further preferably comprises the steps of (i) converting the dicarboxylic acid to its dichloride, and (ii) reacting the dicarboxylic acid dichloride with a diamine compound to obtain an alternating co-oligomer or co-polymer.

The invention will be illustrated by the following, non-limiting examples:

### EXAMPLE 1

### Semi Continuous Reaction for Producing Pentenoic Acid from Butadiene

A 1.2 l mechanically stirred autoclave was charged with 130 g pentenoic acid, 1.55 g water and 10 g tetradecane. The autoclave was flushed three times with CO at 3.0 MPa. Then the autoclave was pressurised with CO to 5.0 MPa, and 5 g of butadiene were added into reactor. Then as the catalyst components, a solution of 0.1 mmol of palladium acetate and 0.3 mmol of 1,2-bis(di-tert-butylphosphinomethyl)benzene dissolved in 10 g pentenoic acid was injected. The injector was rinsed with a further 10 g of pentenoic acid.

Then butadiene and water were continuously added under stirring to the reactor at a rate of 60 mmol/h, while the reactor was heated to 140° C. over 30 minutes. When this temperature was reached, the pressure was adjusted to 8.0 MPa, and the reactor maintained under these conditions for 45 hours, and samples were taken at regular intervals. During this reaction the water concentration was maintained at approximately 1% w/w of the reactor medium. After 45 hours, the butadiene feed was stopped.

After cooling and release of the pressure, the contents of the autoclave were analysed with GLC. The turn over number (TON) of the reaction was calculated as 16,000 mol pentenoic acid/mol catalyst.

### COMPARATIVE EXAMPLE 1

### Semi Continuous Reaction for Producing Pentenoic Acid from Butadiene

Example 1 was repeated, however 5.19 g water were added to the reactor instead of 1.55 g, and subsequently the water concentration was maintained at approximately 3% w/w of the reactor medium. The TON of the reaction was determined as 8,000 mol pentenoic acid/mol catalyst.

### EXAMPLE 2

### Semi Continuous Reaction for In-Situ Conversion of Esters of Butadiene and Pentenoic Acid

A 1.2 l mechanically stirred autoclave was charged with 165 g pentenoic acid, 30 g adipic acid and 3.8 g tetradecane. The autoclave was flushed three times with carbon monoxide at 3.0 MPa. Then the autoclave was pressurised with carbon monoxide to 1.0 MPa, and 25 g of butadiene were added. Then as the catalyst system, a solution of 0.5 mmol of palladium acetate and 1.0 mmol of 1,2-bis(di-tert-butylphosphino-methyl)benzene dissolved in 10 g pentenoic acid was injected into the reactor. The injector was rinsed with a further 10 g of pentenoic acid. Then butadiene was continuously added to the reactor at a rate of 125 mmol/h, while the reactor was heated to 105° C. over 30 minutes. When this temperature was reached, the pressure was adjusted to 8.0 MPa. The reactor was maintained under stirring under these conditions were maintained for 15 hours, and samples taken at regular intervals. Once a TON of 1150 mol esters/mol catalyst was determined, the butadiene feed was stopped, and the pressure released. Then carbon monoxide was bubbled through the reactor at atmospheric pressure for approximately 5 hours, and at regular intervals, samples were taken. After cooling, the contents of the autoclave after the reaction were analysed with gas liquid chromatography (GLC), as were the samples taken. It was found that the esters of pentenoic acid and butadiene had been converted to butadiene and pentenoic acid at a turn over frequency (TOF) of approximately 80 mol esters/mol palladium/hour. The obtained mixture was subjected to a further carbonylation under the conditions as set out above in Example 1, i.e. reactor temperature of 105° C. and carbon monoxide pressure adjusted to 8.0 MPa, however maintaining a water concentration of 7% (w/w). Adipic acid was obtained in an overall selectivity starting from butadiene of about 95%.

The examples clearly show that the combination of maintaining a low water concentration in the first reaction step, while maintaining a high water concentration in the second step allows to obtain adipic acid in high purity and with an overall high turn over frequency, which makes the present process suitable for a continuous industrial process.

1. A process for the preparation of a dicarboxylic acid, comprising the steps of
(a) contacting a conjugated diene with carbon monoxide and water to obtain a mixture containing an ethylenically unsaturated acid product and reversible adducts formed by the conjugated diene with the ethylenically unsaturated acid; and
(b) reacting the ethylenically unsaturated acid product further with carbon monoxide and water to obtain the dicarboxylic acid,
wherein step (a) and (b) are performed in the presence of a catalyst system including a source of palladium, a source of an anion and a bidentate phosphine ligand, and wherein in step (a) the water concentration is maintained at a range of from 0.001 to less than 3% by weight of water, calculated on the overall weight of the liquid reaction medium, and wherein in step (b) the water concentration is maintained at a range of from 3% to 50% by weight of water, calculated on the overall weight of the liquid reaction medium.

2. The process of claim 1, wherein step (a) is conducted until at least 99.99% of the conjugated diene is converted, and wherein the obtained mixture comprising the ethylenically unsaturated acid is directly subjected to step (b).

3. The process of claim 1, wherein step (a) is conducted until at most 99% of the conjugated diene is converted, and wherein step (a) is followed by a step (a1) for the removal of the conjugated diene and reversible diene adducts of the conjugated diene and the ethylenically unsaturated acid formed in step (a), and the mixture depleted of the conjugated diene and the reversible diene adducts is subjected to step (b).

4. The process of claim 1, further comprising a step (c) of separating the saturated dicarboxylic acid product from the reaction mixture obtained in step (b) to obtain a fraction comprising at least part of the catalyst, and recycling of the fraction comprising at least part of the catalyst obtained in step (c) to step (a).

5. The process of claim 4, wherein water is removed from the catalyst fraction prior to recycling to step (a).

6. The process of claim 3, wherein the conjugated diene and reversible diene adducts removed from the reaction mixture obtained in step (a) are recycled to step (a).

7. The process of claim 1, wherein the conjugated diene is 1,3-butadiene.

8. The process of claim 1, wherein the ethylenically unsaturated acid product of step (a) is employed as solvent for the process.

9. The process claim 1, wherein the bidentate diphosphine ligand of formula R¹R²P—R—PR³R⁴ is employed, in which ligand R represents a divalent organic bridging group, and R¹, R², R³ and R⁴ each represent an organic group that is connected to the phosphorus atom through a tertiary carbon atom.

10. The process of 1, wherein the steps (a) and (b) are performed continuously.

11. The process of claim 1, wherein the catalyst system is present in step (a) in an amount of at least 20 ppm, calculated on the total of liquid reaction medium.

12. The process of claim 1, further comprising a step of purifying the dicarboxylic acid.

13. The process of claim 1, further comprising the steps of
(i) converting the dicarboxylic acid to its dichloride, and
(ii) reacting the dicarboxylic acid dichloride with a diamine compound to obtain an alternating co-oligomer or co-polymer.
